(19) United States
(12) Patent Application Publication
(10) Pub. No.: US 2006/0282046 A1
(43) Pub. Date: Dec. 14, 2006

(54) Device and method for subcutaneous delivery of blood clotting agent

(76) Inventors: Jeffrey L. Horn, Rocky Hill, CT (US); Raymond J. Huey, Orange, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/404,126

(22) Filed: Apr. 13, 2006

Related U.S. Application Data
(60) Provisional application No. 60/671,192, filed on Apr. 13, 2005.

(51) Int. Cl. A61M 25/00

(52) U.S. Cl. 604/264

(57) ABSTRACT

A device for promoting the clotting of blood at a wound site in a blood vessel includes a catheter and a blood clotting agent dispensable from the catheter. The blood clotting agent is fed via the catheter to the wound site. A device for promoting the clotting of blood at an access point includes a tube defined by at least one wall, a dispensing lumen formed in the wall, a dispensing port formed at a forward end of the dispensing lumen and at an outside surface of the wall, and a blood clotting agent dispensable from the dispensing port. A method for delivering a blood clotting agent to a wound generally located in a blood vessel includes inserting a sheath through an opening in the patient's flesh and into a blood vessel and dispensing the blood clotting agent to the interface between the flesh and the blood vessel.

### CROSS REFERENCE TO RELATED APPLICATIONS

This application claims the benefits of U.S. Provisional Patent Application Ser. No. 60/671,192, filed on Apr. 13, 2005, and U.S. patent application Ser. No. 10/961,604, filed on Oct. 12, 2004, the contents of both applications being incorporated by reference herein in their entireties.

### TECHNICAL FIELD

The present invention relates generally to blood clotting devices and, more particularly, to blood clotting materials, devices incorporating such materials, and methods for the subcutaneous delivery of such materials for controlling bleeding subsequent to catheterization and like procedures.

### BACKGROUND OF THE INVENTION

Blood is a liquid tissue that includes red cells, white cells, corpuscles, and platelets dispersed in a liquid phase. The liquid phase is plasma, which includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of surgical procedures, the cessation of blood flow is important to the success of the procedure. For example, percutaneous transluminal coronary angioplasty (PTCA) is presently a viable alternative to bypass surgery for the revascularization of stenotic and occluded (e.g., collapsed) coronary arteries and further has applications in the treatment of peripheral artery disease. The PTCA procedure is performed through a puncture made in the patient's aorta. A guidewire is inserted into the aorta and advanced to the desired location in the coronary artery orifice. A catheter is then inserted into the aorta and into the coronary artery orifice via the guidewire. The catheter typically includes a selectively-inflatable balloon of a predetermined size that is negotiated through the aorta to the site of the stenosis where the balloon is cyclically inflated and deflated multiple times. (Cyclic inflation and deflation is necessary to allow blood flow between the inflations.) Upon inflation of the balloon, the vessel at the site of the stenosis is made to dilate. Once the vessel itself is dilated and stable, the balloon is deflated and withdrawn from the site. When the balloon is withdrawn back to the point of insertion into the patient, the balloon and the catheter are retracted from the patient through the puncture.

Because of the nature of PTCA (or any arterially-invasive surgical procedure), retraction of the catheter through the puncture can have an adverse effect on the closing of the puncture. More specifically, because the puncture is made directly in a blood vessel, subcutaneous bleeding may occur. Subcutaneous bleeding is the leakage of blood from the vessel and its migration along the interfacial area between the outer surface of the vessel and an adjacent surface of the surrounding tissue. At best, the result is a minor pooling of blood beneath the surface of the skin to form a bruise. At worst, the puncture into the blood vessel does not close and severe blood loss occurs.

In an effort to address the above-described problem, devices have been developed for inhibiting bleeding upon retraction of a catheter. Some devices include patches and plug members that are retained in the puncture as the catheter is withdrawn from the patient. Other devices include materials (e.g., silicone or polyurethane) that are coated onto the tips of catheters such that upon withdrawal of the catheter, properties of the materials facilitate the self-occlusion of the puncture site. Although these materials have been shown to be somewhat successful, they add a level of complexity to the surgical procedure, can be rejected by the host body, and can otherwise be ineffective in some situations.

Based on the foregoing, it is a general object of the present invention to provide devices for controlling subcutaneous bleeding and methods of their use that overcome or improve upon the prior art.

### SUMMARY OF THE PRESENT INVENTION

In one aspect, the present invention is directed to a device for promoting the clotting of blood at a wound site in an artery during PTCA procedures. The device includes a catheter defined by a body and a guide positioned at a forward end of the body as well as a blood clotting agent that can be dispensed from the catheter. To dispense the blood clotting agent, a longitudinally extending passage is formed in the wall of the catheter, and the blood clotting agent is fed through or from a lumen defined by the wall of the catheter to the wound site. In a second aspect, the present invention is directed to a device for promoting the clotting of blood at an access point of a patient (e.g., the point at which a catheter is inserted into the aorta during a PTCA procedure). The device includes a tube defined by at least one wall, a dispensing lumen formed in the wall, a dispensing port formed at a forward end of the dispensing lumen and at an outside surface of the wall, and a blood clotting agent dispensable from the dispensing port. The dispensing lumen is configured to transfer the blood clotting agent to the dispensing port, and the dispensing port is configured to dispense the blood clotting agent to the bleed site.

In a third aspect, the present invention is directed to a method for delivering a blood clotting agent to a subcutaneous wound generally located in a blood vessel, such wounds often being from catheterization procedures. The method includes inserting a sheath through an opening in the patient's flesh and into a blood vessel and dispensing the blood clotting agent to the interface between the flesh and the blood vessel. The sheath includes a tube defined by at least one wall, a dispensing lumen formed in the wall, and a dispensing port formed at a forward end of the dispensing lumen at an outside surface of the wall.

One advantage of the present invention is that wounds in blood vessels can be treated from inside the vessels themselves before the termination of a PTCA or similar procedure. Thus, as the wound can be treated before all devices are retracted from the patient's body, the need for gaining access to the blood vessel twice can be eliminated.

Another advantage of the present invention is that an opening formed in a blood vessel for purposes relating to catheterization may be closed by clotting the blood at a precise point, viz., at the vessel wall. By clotting the blood at the vessel wall, the pressure typically applied to the area from which the catheter is withdrawn to stop bleeding can be reduced or eliminated altogether. Reducing the amount of pressure or eliminating the need for pressure altogether correspondingly reduces or eliminates the discomfort typically experienced in procedures in which catheterization points must be closed and also minimizes the potential for further damage at the wound site.

Still another advantage is that clotting can be initiated during the catheterization itself to reduce or eliminate the leakage of blood at the access point during the catheterization. More particularly, by dispensing the blood clotting agent through the dispensing lumen of a sheath through which a catheter is inserted, blood leakage from around the sheath can be slowed or stopped. Thus, the pooling of blood around a catheterization site is minimized.

### BRIEF DESCRIPTION OF THE DRAWINGS

FIG. 1 is a schematic representation of a first embodiment of a catheter system of the present invention.

FIG. 2 is a schematic representation of the body of the catheter system of FIG. 1.

FIG. 3 is a schematic representation of a portion of the catheter system through which a blood clotting agent is dispensed.

FIG. 4 is a schematic representation of a second embodiment of the catheter system of the present invention.

### DETAILED DESCRIPTION OF THE PREFERRED EMBODIMENT

Disclosed herein are devices and methods for delivering materials to interface regions of tissue and blood vessels to promote the clotting of blood and to limit the degree of subcutaneous bleeding during arterially-invasive procedures such as transluminal angioplasty and the like. The devices generally comprise catheter systems that are capable of delivering blood clotting agents to the walls of a blood vessel and, more particularly, to the opening formed in the vessel wall for the purpose of inserting a catheter. The systems minimize or stop subcutaneous bleeding upon removal of the catheter by absorbing at least portions of the liquid phases of the blood, thereby promoting clotting at the interface of the tissue and vessel in the region of the opening formed by the catheter. One exemplary blood clotting agent that can be used with the devices and methods of the present invention is molecular sieve material. Another exemplary material that can be used with the devices and methods of the present invention as the blood clotting agent is oxidized cellulose.

The molecular sieve material may be a synthetic polymer gel, porous silica gel, porous glass, alumina, hydroxyapatite, calcium silicate, zirconia, zeolite, or the like. Exemplary synthetic polymers include, but are not limited to, stylene-divinylbenzene copolymer, cross-linked polyvinyl alcohol, cross-linked polyacrylate, cross-linked vinyl ether-maleic anhydride copolymer, cross-linked stylene-maleic anhydride copolymer or cross-linked polyamide, and combinations thereof.

The molecular sieve material is preferably a zeolite. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. Typically, the zeolite is a friable material that is about 90% by weight calcium and about 10% by weight sodium. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings.

The zeolite may be mixed with or otherwise used in conjunction with other materials having the ability to be dehydrated without significant changes in crystalline structure. Such materials include, but are not limited to, magnesium sulfate, sodium metaphosphate, calcium chloride, dextrin, a polysaccharide, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

The oxidized cellulose may be cellulosic acid, absorbable cellulose, polyanhydroglucuronic acid, or any chemically oxidized form of cellulose fiber. Oxidized cellulose is cellulose that has an increased carboxylation content due to variation in the degree of oxidation. The cellulose fiber that may be used in the present invention may be any common cellulose fiber such as cotton.

Oxidized cellulose may be manufactured by the action of nitrogen dioxide gas on cellulose fiber. Other methods of manufacturing oxidized cellulose include oxidation of cellulose fiber with aqueous oxidizing agents such as hypochlorite salts, although the use of such agents is less preferred than the use of nitrogen dioxide gas.

One method of generating nitrogen dioxide gas is by the catalytic reaction of manganese dioxide or manganese disulfide on concentrated nitric acid. Any amount of nitrogen dioxide can be generated by the metered addition of nitric acid to the manganese dioxide or manganese disulfide catalyst. In such a reaction, dinitrogen tetroxide is also formed in addition to the nitrogen dioxide. The formation of dinitrogen tetroxide does not have an interfering effect on the oxidation of the cellulose.

In this method of nitrogen dioxide generation, unaltered cellulose fibers are introduced into a reaction vessel, and concentrated nitric acid is metered into a second enclosed vessel containing manganese dioxide powder. Nitrogen dioxide gas is evolved, which is piped to the reaction vessel containing the cellulose fibers. Once the nitrogen dioxide gas is piped to the reaction vessel containing the cellulose fibers, the reaction vessel is purged with an excess amount of nitrogen dioxide and left sealed for 36 hours. The oxidized cellulose is then removed and washed in dilute sodium bicarbonate solution, followed by multiple agitated rinses with distilled water. The resulting oxidized cellulose is thus sufficiently carboxylated to provide a desirable hemostatic effect on a bleeding wound. The resulting fibers can also be autoclaved before use.

Another method of generating nitrogen dioxide gas is by the reaction of formaldehyde with concentrated nitric acid. In particular, formaldehyde is consumed in the reaction and is thus depleted.

Various materials may be mixed with, associated with, or incorporated into the zeolites or the oxidized cellulose to maintain an antiseptic environment at the catheterization site or to provide functions that are supplemental to the clotting functions of the zeolites or the oxidized cellulose. Exemplary materials that can be used include, but are not limited to, pharmaceutically-active compositions such as antibiotics, antifungal agents, antimicrobial agents, anti-inflammatory agents, analgesics (e.g., cimetidine, chloropheniramine maleate, diphenhydramine hydrochloride, and promethazine hydrochloride), compounds containing silver ions, and the like. Other materials that can be incorporated to provide additional hemostatic functions include ascorbic acid, tranexamic acid, rutin, and thrombin. Botanical agents having desirable effects on the wound site may also be added.

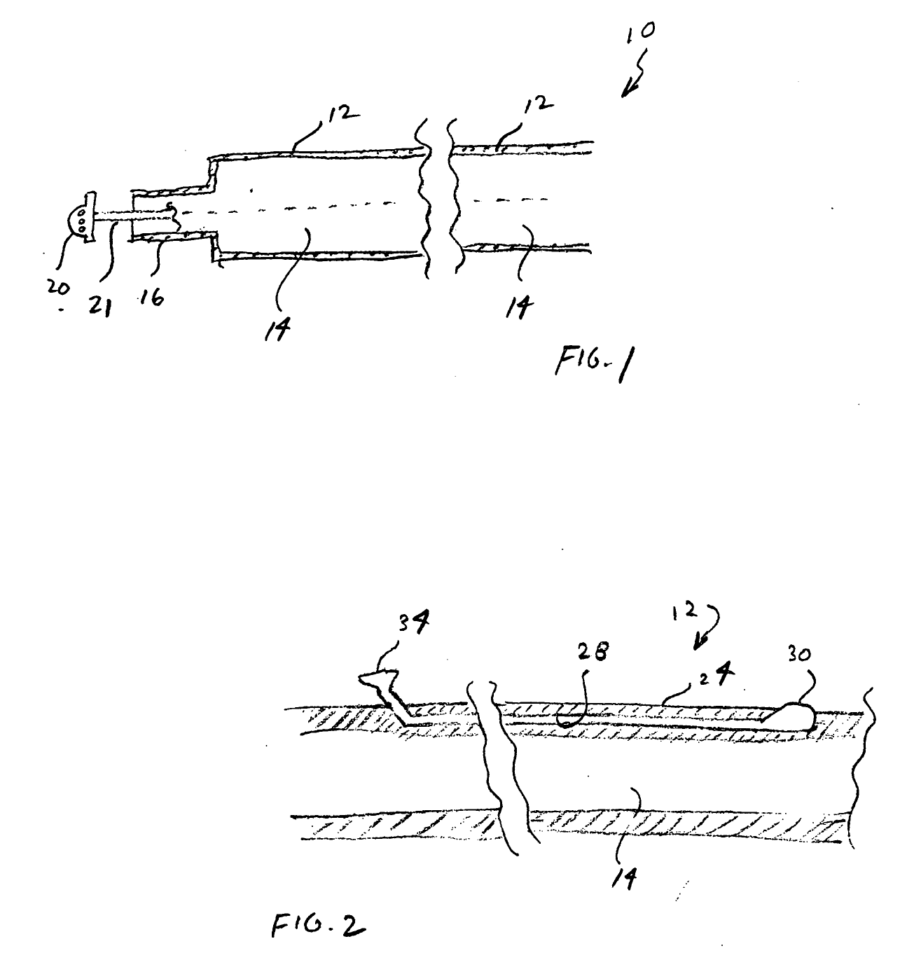

In one embodiment of the present invention, a device for delivering a blood clotting agent directly at a blood vessel puncture site upon termination of an arterially-invasive procedure is the catheter system shown generally at 10 with reference to FIG. 1. The catheter system 10 comprises a body 12 having an axial lumen 14 that defines a channel. The body 12 has at its forward end a tip 16 coaxially formed with the axial lumen 14 to facilitate the negotiation of the catheter system 10 through the blood vessel. A retractable guide 20 is positioned at the forward end of the tip 16 on a wire 21 to facilitate the movement of the tip 16 and body 12 through the vessel. Preferably, the guide 20 is rounded at its forward end or similarly configured to facilitate the penetration of narrow passages and valves of the heart of the patient. Both the guide 20 and the tip 16 are fenestrated to accommodate the transfer of fluids relative to movement of the catheter system 10 through the patient.

Referring now to FIG. 2, the body 12 is defined by a wall 24, the enclosure of which defines the axial lumen 14. A second lumen 28 is formed within the wall 24 and extends longitudinally over the length of the body 12 a sufficient distance to provide communication between a flow control device 30 at a forward portion of the body 12 and a control port 34 at a rearward portion of the body 12. The flow control device 30 is preferably a selectively-inflatable balloon that, when inflated through the control port 34, provides an action that dilates a blood vessel into which the catheter system is inserted. In embodiments of the present invention in which the flow control device 30 is a balloon, the balloon may be annular to uniformly dilate the vessel around the body 12. The balloon (or other type of flow control device 30) may be actuated via the control port 34 using any type of actuation means including, but not limited to, hydraulic, mechanical, electrical, thermal, pressurized gas, combinations of the foregoing means, and the like.

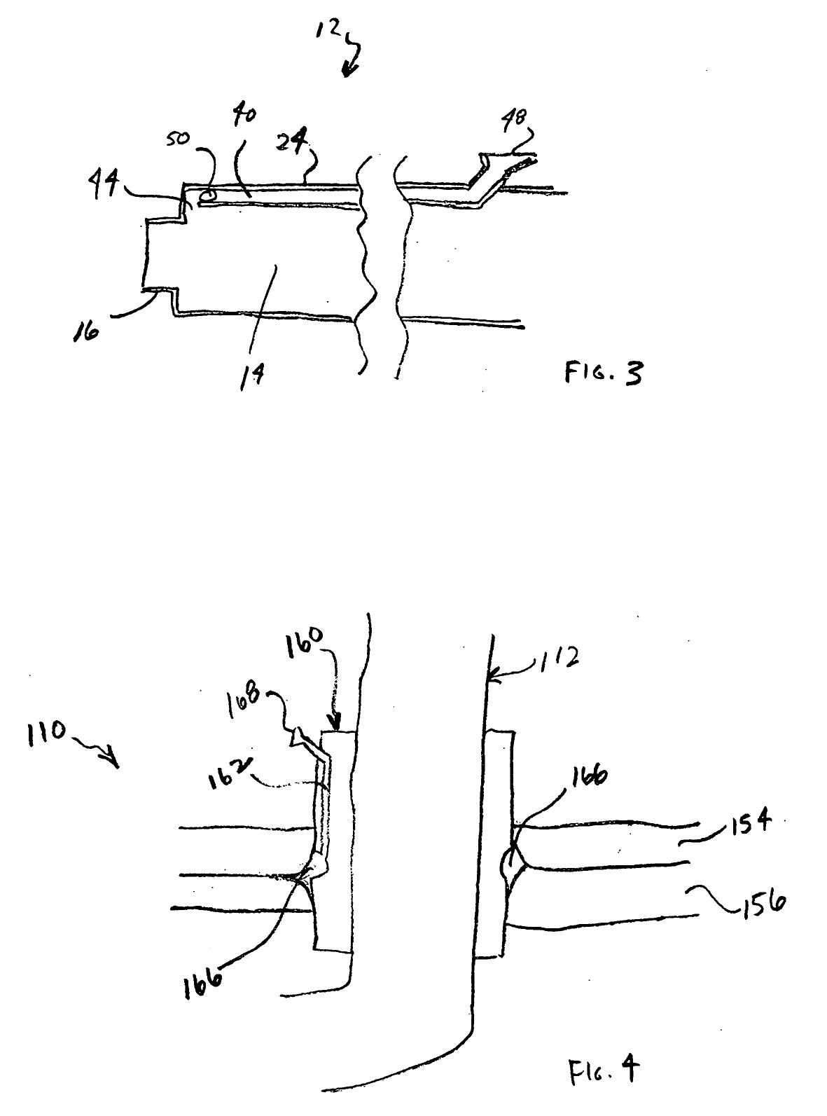

Referring now to FIG. 3, the forward portion of the body 12 proximate the tip 16 is shown. The body 12 includes a dispensing lumen 40 formed in the wall 24. The dispensing lumen 40 extends over the length of the body 12 to provide communication between a dispensing port 44 at the forward end of the body 12 and a dispensing chamber 48 at the rearward end of the body 12. The dispensing chamber 48 is sized to receive a blood clotting agent (such as a zeolite or an oxidized cellulose) therethrough and to facilitate the transfer of the agent to the dispensing port 44. The blood clotting agent is in the form of a discrete particle 50. The discrete particle 50 may be a pellet, a granule, or a bead. The particle 50 may be of a particular size such that a collection of particles defines a powder. Mechanical or fluid means may be used to transfer the particle 50 through the dispensing lumen 40. The dispensing port 44 is adjacent or proximate the tip 16 and adjacent the axial lumen 14 and is sized to dispel the particle 50 into the forward portion of the axial lumen 14 and into the tip 16. A positive pressure (relative to the pressure outside the forward end of the tip 16) may be maintained in the axial lumen 14 to dispel the particle 50 into the opening of the puncture upon retraction of the catheter system from the blood vessel.

Irrespective of the type of blood clotting agent (zeolite or oxidized cellulose), the particles 50 in the preferred embodiments of the present invention exhibit a median particle size of about 7 microns. The present invention is not limited in this regard, however, as particles of other sizes can be substituted for those described herein without departing from the broader aspects of the present invention.

To operate the catheter system 10 of FIGS. 1-3, a puncture is made through the tissue of the patient and into the blood vessel. The catheter system 10 is inserted and operated to perform the necessary procedure. Upon completion of the procedure, the catheter system 10 is retracted until the forward-most portion of the tip 16 is positioned substantially at the puncture site. The particle 50, which may be pre-loaded into the dispensing chamber 48, is transferred through the dispensing port 44 using any suitable means (e.g., mechanically, using pressurized fluid, or the like). A suitable pressure is maintained in the axial lumen 14 to force the particle 50 out of the tip 16 where it is captured by the edges that define the puncture.

In another embodiment of the present invention as shown with reference to FIG. 4, a catheter system is shown generally at 110. The catheter system 110 comprises a body 112 through which typical catheter operations can be performed when the system is inserted through a puncture opening in a tissue 154 of a patient and through a wall of a blood vessel 156 into a lumen of the blood vessel 156. The catheter system 110 also comprises a sheath 160 through which the body 112 is able to freely slide.

The sheath 160 is a tubular structure having at least one dispensing lumen 162 formed in a wall thereof that provides communication between a dispensing port 166 and a dispensing chamber 168. Preferably, the dispensing port 166 extends annularly around the outer surface of the sheath 160. The dispensing lumen 162, the dispensing port 166, and the dispensing chamber 168 are each sized to accommodate the flow of blood clotting agent (e.g., zeolite, oxidized cellulose, or a combination thereof) therethrough.

To operate the catheter system 110, a puncture is made through the tissue of the patient and into the blood vessel. The sheath 160 is inserted into the puncture, and the dispensing port(s) 166 of the sheath 160 are aligned with the interface of the tissue and the blood vessel. The body 112 is inserted through the sheath 160 and operated as necessary to perform the desired procedure. During the procedure, blood clotting agent in the form of pellets or powder is fed through the dispensing lumen 162 either intermittently or at a constant rate to provide the desired clotting effect at the tissue/blood vessel interface. Once the body 112 is retracted from the blood vessel and the sheath 160, the sheath can be removed from the puncture.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A device for promoting the clotting of blood at a wound site in a blood vessel, said device comprising:
a catheter, comprising,
a body defined by a wall and having a forward end,
a guide positioned at said forward end of said body, and
a dispensing lumen formed in said wall; and
a blood clotting agent dispensable through said dispensing lumen to a wound site.

2. The device of claim 1, further comprising a dispensing port at a forward end of said dispensing lumen and a dispensing chamber proximate a rearward end of said dispensing lumen, said dispensing lumen extending over a length of said body to provide communication between said dispensing port and said dispensing chamber.

3. The device of claim 1, wherein said blood clotting agent is a discrete particle.

4. The device of claim 1, wherein said blood clotting agent is selected from the group consisting of zeolites, oxidized celluloses, and combinations of the foregoing.

5. The device of claim 4, wherein said blood clotting agent further comprises an antibiotic, an antifungal agent, an antimicrobial agent, an anti-inflammatory agent, an analgesic, a compound containing silver ions, ascorbic acid, tranexamic acid, rutin, thrombin, a botanical agent, or a combination of the foregoing.

6. The device of claim 1, further comprising a flow control device incorporated into said catheter at said forward end of said body.

7. The device of claim 6, wherein said flow control device is a selectively inflatable balloon.

8. The device of claim 7, wherein said balloon is annularly attached around said body of said catheter.

9. The device of claim 1, wherein said guide includes a wire attached thereto, said wire being capable of facilitating the retraction of said guide.

10. A device for promoting the clotting of blood at an access point of a patient, said device comprising:
a tube defined by at least one wall, said tube being insertable into an opening in tissue and through a wall of a blood vessel;
a dispensing lumen formed in said wall of said tube;
a dispensing port formed at a forward end of said dispensing lumen at an outside surface of said wall of said tube; and
a blood clotting agent dispensable from said dispensing port.

11. The device of claim 10, further comprising a dispensing chamber formed at a rearward end of said dispensing lumen, said dispensing chamber being in communication with said dispensing lumen to receive said blood clotting agent and to transfer said blood clotting agent to said dispensing lumen.

12. The device of claim 10, further comprising a catheter inserted through said tube into said blood vessel.

13. The device of claim 10, wherein said dispensing port extends annularly around said outside wall of said tube.

14. The device of claim 10, wherein said blood clotting agent is selected from the group consisting of zeolites, oxidized celluloses, and combinations of the foregoing.

15. The device of claim 14, wherein said blood clotting agent further comprises an antibiotic, an antifungal agent, an antimicrobial agent, an anti-inflammatory agent, an analgesic, a compound containing silver ions, ascorbic acid, tranexamic acid, rutin, thrombin, a botanical agent, or a combination of the foregoing.

16. A method for delivering a blood clotting agent to a wound in a blood vessel, said method comprising the steps of:
inserting a sheath into a blood vessel, said sheath comprising,
a tube defined by at least one wall,
a dispensing lumen formed in said wall of said tube, and
a dispensing port formed at a forward end of said dispensing lumen at an outside of said wall of said tube; and
dispensing a blood clotting agent from said dispensing port to an interface between said blood vessel and adjacent tissue.

17. The method of claim 16, wherein said blood clotting agent is selected from the group consisting of zeolites, oxidized celluloses, and combinations of the foregoing.

18. The method of claim 16, further comprising puncturing said adjacent tissue to form an opening in said tissue.

19. The method of claim 16, further comprising aligning said dispensing port with said interface between said blood vessel and said adjacent tissue.

20. The method of claim 16, further comprising inserting a catheter through said sheath.

21. The method of claim 16, further comprising removing said sheath from said blood vessel.
